(19) United States
(12) Patent Application Publication
(10) Pub. No.: US 2026/0250245 A1
(43) Pub. Date: Aug. 27, 2026

(54) ALKENYL SUCCINIMIDE COMPOUNDS AND THEIR USE

(71) Applicant: VERTELLUS HOLDINGS LLC, Indianapolis, IN (US)

(72) Inventors: Brett DUKE, Denham Springs, LA (US); Atanu ADHVARYU, Glen Allen, VA (US); Matthew STEPHENS, Cranston, RI (US)

(21) Appl. No.: 19/476,383

(22) PCT Filed: Mar. 28, 2023
(86) PCT No.: PCT/US2023/016576

§ 371 Date: Oct. 17, 2025

Related U.S. Application Data
(60) Provisional application No. 63/324,918, filed on Mar. 29, 2022.
(60) Provisional application No. 63/454,515, filed on Mar. 24, 2023.

(51) Int. Cl. C07D 207/408

(52) U.S. Cl. CPC C07D 207/408

(57) ABSTRACT

This disclosure relates to processes for preparing succinimide derivatives that are useful in the chemical arts, such as in the manufacture of products, such as lubricants and fuel additives. In particular, the present disclosure pertains to novel processes for preparing certain succinimide derivatives.

### CROSS-REFERENCE TO RELATED APPLICATIONS

This application claims priority under 35 U.S.C. § 119(e) to U.S. Provisional Application Ser. No. 63/454,515 filed on Mar. 24, 2023 and U.S. Provisional Application Ser. No. 63/324,918 filed on Mar. 29, 2022, the entire disclosures of which are incorporated herein by reference.

### TECHNICAL FIELD

This disclosure relates to processes for preparing succinimide derivatives that are useful in the chemical arts, such as in the manufacture of products, such as lubricants and fuel additives. In particular, the present disclosure pertains to novel processes for preparing certain succinimide derivatives.

### BACKGROUND

In modern automotive fuels, one or more chemical additives, often several in combination, are used in order for the fuel to meet desired performance levels. Specifically, chemical additives in small dosages combine to add or improve properties of virgin fuels that cannot be obtained through the refining processes. There are a number of reasons for using additives in fuels, such as, improving handling properties and stability of fuel; improving combustion properties of fuel; reducing emissions from fuel combustion; providing engine protection and cleanliness; and increasing the economic use of fuel. Some of the most important additives used in the market are those that improve the flow of gasoline and diesel oils (i.e. lubricity improvers) and provide engine protection and cleanliness (i.e. corrosion inhibitors).

Due to the widespread introduction of low sulfur fuel, inherent lubricity of fuel is significantly reduced, therefore compromising wear and deposit control. Lubricity is a critical parameter of fuel quality and is important to maintain proper functioning of the fuel injection system hardware.

Lubricity improvers work through the action of film formation on the metal surfaces. Lubricity improvers are meant for protecting the fuel pump from wear through the mechanism of surface adsorption. Lubricity improvers are generally surface active compounds and get concentrated at the surfaces of separation, forming extremely thin adsorption layers. These thin layers are capable of producing marked changes in molecular nature and surface characteristics. This leads to a change in the kinetics of the processes involved in the transfer of substances across surfaces of separation and in the second place to the changes in the condition of molecular interaction between the two contacting surfaces. Examples of lubricity improvers include compositions of polybasic acid or a polybasic acid ester, with C₁-C₅ monohydric alcohols with a partial ester of a polyhydric alcohol and a fatty acid; reaction products of a dicarboxylic acid and an oil-insoluble glycol; and salts of a carboxylic acid and an aliphatic amine or an amide obtained by dehydration-condensation. Corrosion is a chemical process where the metal undergoes oxidation with the gradual degradation and deterioration of the metal surface. It is a common failure mode observed with fuel and lubricant applications and can be prevented by using suitable chemistry to passivate the metal surface.

Corrosion inhibitors are useful for providing engine protection. Trace contaminations of commercial gasolines with water cannot be avoided. Sometimes the moisture may be picked up from the atmosphere. This moisture along with air (oxygen) can attack iron and other metal in storage tanks, pipelines, tankers, and fuel tanks of automobiles, leading to severe corrosion problems. In addition, rust particles can clog fuel filters and carburetor/injectors orifices and adversely affect engine performance. The extent of rusting depends on the temperature, humidity, exposure to the environment, and their duration. Corrosion is the outcome of the reaction of acidic compounds on metals. Corrosion inhibitors find use in combating these issues. Examples of corrosion inhibitors include high molecular weight carboxylic, sulphonic, or phosphoric acids; salts of these acids; and products of neutralization of the acids with organic bases such as amines. Alkyl succinates and alkylene succinic anhydride acid derivatives have been used in lubricants as corrosion inhibitors and in additives in engine oils, demonstrated as early as in the 1960s (See, Srivastava, S. P. Fuels and Fuel Additives, Chapt. 5, 2014, John Wiley & Sons, Inc.). The use of succinimides has also been reported (See, U.S. Pat. No. 5,411,559, incorporated herein by reference).

With the rise of hybrid and electric vehicles (EVs), the automotive industry is going through a profound shift. There is a need to manage the challenges and meet the technical requirements placed on fluids in hybrids and electric vehicles. One or more chemical additives, often several in combination, are used in order for the electric vehicle fluids to meet desired performance levels. Specifically, chemical additives in small dosages combine to add or improve properties of the fluid such as to improve the reliability, efficiency and performance for electric powertrains. Fluid additives can be used in consumer vehicles such as EVs, through to commercial vehicles such as electric in-city delivery vans and hydrogen buses and long range trucks. Because hybrids and electric vehicles continue to deliver a higher level of performance for consumers, there is a continued need to provide more advanced, reliable e-fluids in the industry. Some of the most important additives used in the market are those that improve the properties in electric transmission fluid, electric thermal fluid, and electric grease.

In addition to lubricity and corrosion, properties of additives, fluids, and fuels that can affect the vehicle performance can include, but are not limited to, electrical conductivity, dielectric breakdown voltage, and oxidative degradation. Electrical conductivity is one of the key parameters to determine the suitability of lubricant used as electric drive fluid (EDF). Lubricants used in an eMotor are designed to deliver appropriate low conductivity to prevent charge build-up and prevent arcing and cause hardware damage. External factors such as temperature, time and moisture content can alter fluid conductivity. Polar components arising from oxidation will also increase the ability of the lubricant to carry charge. For optimal performance, EDF is expected to deliver low and stable electrical conductivity over the operational life of the lubricant. Dielectric breakdown voltage is a measure of the resistance of an insulator to break down under an applied electric field. This breakdown of the insulation often results in arcing which can lead to hardware failure in high voltage applications. Oxidative degradation of fuels and oils results in insoluble and soluble materials that are the precursors of engine deposit and lacquers often observed in high temperature applications.

With the advent of higher performing traditional and electric vehicles, the demand for chemical additives has steadily increased in recent years. The reported synthetic methods to make succinimide derivatives, such as hexadecenyl (C16 alkenyl) succinimide, have been either non-economical and/or generate too much waste to become a commercially relevant manufacturing process for the larger volumes required in manufacture of products, such as lubricants, fluid additives, and fuel additives. Accordingly, there remains a need for improved processes and intermediates for use in the preparation of succinimide derivatives.

### SUMMARY

In one aspect, the present disclosure provides a compound of the formula I

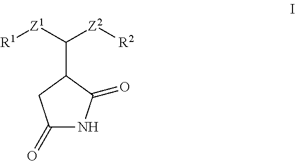

wherein
Z¹ and Z² are each independently a bond or —CH═CH—, provided that one of Z¹ or Z² is —CH═CH—, and one of Z¹ or Z² is a bond;
R¹ is H or C₁-C₆ alkyl; and
R² is C₇-C₃₆ alkyl.

In another aspect, the present disclosure provides a composition comprising of a compound of the formula I

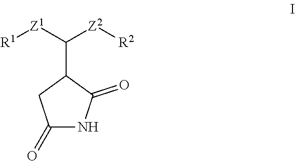

wherein
Z¹ and Z² are each independently a bond or —CH═CH—, provided that one of Z¹ or Z² is —CH═CH—, and one of Z¹ or Z² is a bond;
R¹ is H or C₁-C₆ alkyl; and
R² is C₇-C₃₆ alkyl.

It has been shown herein that the compounds and compositions described herein provide both very good lubricity properties and corrosion resistance.

In another aspect, the present disclosure provides a process for preparing a compound of the formula I

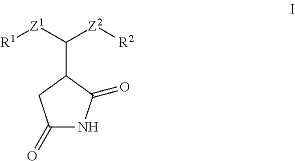

wherein
Z¹ and Z² are each independently a bond or —CH═CH—, provided that one of Z¹ or Z² is —CH═CH—, and one of Z¹ or Z² is a bond;
R¹ is H or C₁-C₆ alkyl; and
R² is C₇-C₃₆ alkyl,
comprising one or more of steps
i. contacting a terminal C₁₀-C₄₄ alkyl olefin with a catalyst under conditions suitable to isomerize the terminal C₁₀-C₄₄ alkyl olefin to one or more of a beta-gamma C₁₀-C₄₄ alkyl olefin, gamma-delta C₁₀-C₄₄ alkyl olefin, delta-epsilon C₁₀-C₄₄ alkyl olefin, epsilon-zeta C₁₀-C₄₄ alkyl olefin, zeta-eta C₁₀-C₄₄ alkyl olefin, or eta-theta C₁₀-C₄₄ alkyl olefin; or
ii. contacting the beta-gamma C₁₀-C₄₄ alkyl olefin, gamma-delta C₁₀-C₄₄ alkyl olefin, delta-epsilon C₁₀-C₄₄ alkyl olefin, epsilon-zeta C₁₀-C₄₄ alkyl olefin, or zeta-eta C₁₀-C₄₄ alkyl olefin product of step (i) with maleic anhydride under conditions suitable to provide a compound of the formula II

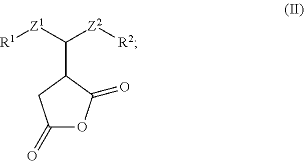

or
iii. contacting a compound of the formula II

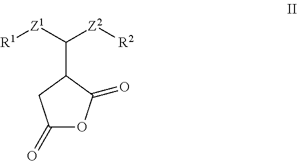

with an ammonia source under conditions sufficient to provide a compound of the formula I.

In another aspect, the present disclosure provides a process for preparing a compound of the formula I

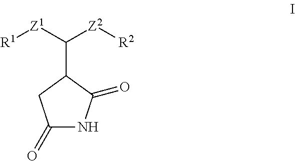

wherein
Z¹ and Z² are each independently a bond or —CH═CH—, provided that one of Z¹ or Z² is —CH═CH—, and one of Z¹ or Z² is a bond;
R¹ is H or C₁-C₆ alkyl; and
R² is C₇-C₃₆ alkyl,
comprising
i. contacting a terminal C₁₀-C₄₄ alkyl olefin with catalyst under conditions suitable to isomerize the terminal C₁₀-C₄₄ alkyl olefin to one or more of a beta-gamma C₁₀-C₄₄ alkyl olefin, gamma-delta C₁₀-C₄₄ alkyl olefin, delta-epsilon C₁₀-C₄₄ alkyl olefin, epsilon-zeta C₁₀-C₄₄ alkyl olefin, zeta-eta C₁₀-C₄₄ alkyl olefin, or eta-theta C₁₀-C₄₄ alkyl olefin; and
ii. contacting the beta-gamma C₁₀-C₄₄ alkyl olefin, gamma-delta C₁₀-C₄₄ alkyl olefin, delta-epsilon C₁₀-C₄₄ alkyl olefin, epsilon-zeta C₁₀-C₄₄ alkyl olefin, or zeta-eta C₁₀-C₄₄ alkyl olefin product of step (i) with maleic anhydride under conditions suitable to provide a compound of the formula II

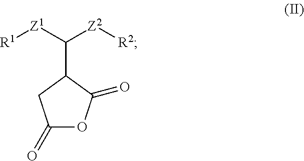

and
iii. contacting a compound of the formula II

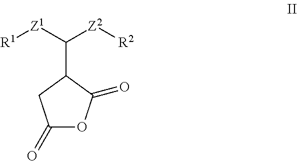

with an ammonia source under conditions sufficient to provide a compound of the formula I.

In another aspect, the present disclosure provides a compound of the formula I

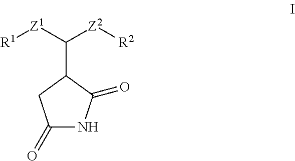

wherein
Z¹ and Z² are each independently a bond or —CH═CH—, provided that one of Z¹ or Z² is —CH═CH—, and one of Z¹ or Z² is a bond;
R¹ is H or C₁-C₆ alkyl; and
R² is C₆-C₃₆ alkyl,
wherein the compound of the formula I is prepared by according to a process as described herein.

In another aspect, the present disclosure provides a composition comprising at least one compound of the formula I

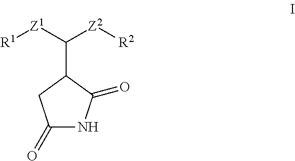

wherein
Z¹ and Z² are each independently a bond or —CH═CH—, provided that one of Z¹ or Z² is —CH═CH—, and one of Z¹ or Z² is a bond;
R¹ is H or C₁-C₆ alkyl; and
R² is C₆-C₃₆ alkyl,
wherein the compound of the formula I is prepared by according to a process as described herein.

Additional embodiments, features, and advantages of the disclosure will be apparent from the following detailed description and through practice of the disclosure. The compounds and methods of the present disclosure can be described as embodiments in any of the following enumerated clauses. It will be understood that any of the embodiments described herein can be used in connection with any other embodiments described herein to the extent that the embodiments do not contradict one another.

1. A compound of the formula I

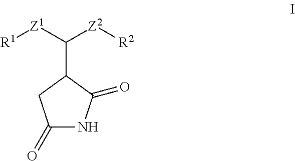

wherein
Z¹ and Z² are each independently a bond or —CH═CH—, provided that one of Z¹ or Z² is —CH═CH—, and one of Z¹ or Z² is a bond;
R¹ is H or C₁-C₆ alkyl; and
R² is C₆-C₃₆ alkyl.

2. The compound of clause 1, wherein the compound is of the formula Ia

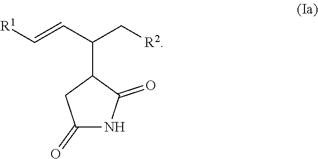

3. The compound of clause 1, wherein the compound is of the formula Ib

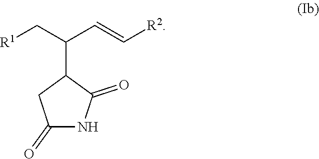

4. The compound of any one of clauses 1 to 3, wherein R¹ is H or C₁-C₅ alkyl.

5. The compound of any one of clauses 1 to 4, wherein R¹ is H, methyl, ethyl, n-propyl, or n-butyl, or n-pentyl.

6. The compound of any one of clauses 1 to 5, wherein R² is C₆-C₃₆ linear alkyl.

7. The compound of any one of clauses 1 to 6, wherein R² is C₈-C₁₄ linear alkyl.

8. The compound of any one of clauses 1 to 5, wherein R² is C₆-C₃₆ branched alkyl.

9. The compound of any one of clauses 1 to 5, or 8, wherein R² is C₈-C₁₄ branched alkyl.

10. A composition comprising of a compound of the formula I

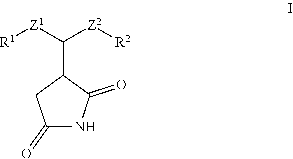

wherein
Z¹ and Z² are each independently a bond or —CH═CH—, provided that one of Z¹ or Z² is —CH═CH—, and one of Z¹ or Z² is a bond;
R¹ is H or C₁-C₆ alkyl; and
R² is C₆-C₃₆ alkyl.

11. The composition of clause 10, wherein the compound is of the formula Ia

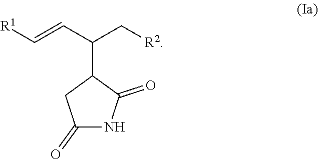

12. The composition of clause 10, wherein the compound is of the formula Ib

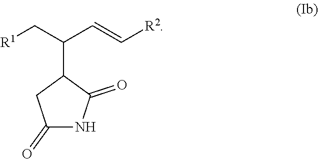

13. The composition of any one of clauses 10 to 12, wherein R¹ is H or C₁-C₅ alkyl.

14. The composition of any one of clauses 10 to 13, wherein R¹ is H, methyl, ethyl, n-propyl, n-butyl, or n-pentyl.

15. The composition of any one of clauses 10 to 14, wherein R² is C₆-C₃₆ linear alkyl.

16. The composition of any one of clauses 10 to 15, wherein R² is C₈-C₁₄ linear alkyl.

17. The composition of any one of clauses 10 to 14, wherein R² is C₆-C₃₆ branched alkyl.

18. The composition of any one of clauses 10 to 14, or 17, wherein R² is C₈-C₁₄ branched alkyl.

19. A process for preparing a compound of the formula I

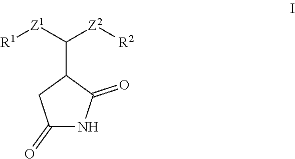

wherein
Z¹ and Z² are each independently a bond or —CH═CH—, provided that one of Z¹ or Z² is —CH═CH—, and one of Z¹ or Z² is a bond;
R¹ is H or C₁-C₆ alkyl; and
R² is C₆-C₃₆ alkyl, comprising one or more of steps
i. contacting a terminal C₁₀-C₄₄ alkyl olefin with a catalyst under conditions suitable to isomerize the terminal C₁₀-C₄₄ alkyl olefin to one or more of a beta-gamma C₁₀-C₄₄ alkyl olefin, gamma-delta C₁₀-C₄₄ alkyl olefin, delta-epsilon C₁₀-C₄₄ alkyl olefin, epsilon-zeta C₁₀-C₄₄ alkyl olefin, zeta-eta C₁₀-C₄₄ alkyl olefin, or eta-theta C₁₀-C₄₄ alkyl olefin; or

ii. contacting the beta-gamma C₁₀-C₄₄ alkyl olefin, gamma-delta C₁₀-C₄₄ alkyl olefin, delta-epsilon C₁₀-C₄₄ alkyl olefin, epsilon-zeta C₁₀-C₄₄ alkyl olefin, or zeta-eta C₁₀-C₄₄ alkyl olefin product of step (i) with maleic anhydride under conditions suitable to provide a compound of the formula II

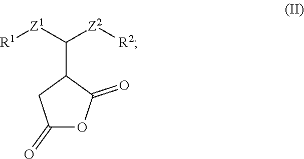

or
iii. contacting a compound of the formula II

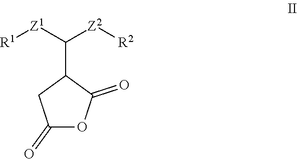

with an ammonia source under conditions sufficient to provide a compound of the formula I.

20. The process of clause 19, comprising
i. contacting a terminal C₁₀-C₄₄ alkyl olefin with a catalyst under conditions suitable to isomerize the terminal C₁₀-C₄₄ alkyl olefin to one or more of a beta-gamma C₁₀-C₄₄ alkyl olefin, gamma-delta C₁₀-C₄₄ alkyl olefin, delta-epsilon C₁₀-C₄₄ alkyl olefin, epsilon-zeta C₁₀-C₄₄ alkyl olefin, zeta-eta C₁₀-C₄₄ alkyl olefin, or eta-theta C₁₀-C₄₄ alkyl olefin.

21. The process of clause 19, comprising
ii. contacting a beta-gamma C₁₀-C₄₄ alkyl olefin, gamma-delta C₁₀-C₄₄ alkyl olefin, delta-epsilon C₁₀-C₄₄ alkyl olefin, epsilon-zeta C₁₀-C₄₄ alkyl olefin, zeta-eta C₁₀-C₄₄ alkyl olefin, or eta-theta C₁₀-C₄₄ alkyl olefin product of step (i) with maleic anhydride under conditions suitable to provide a compound of the formula II

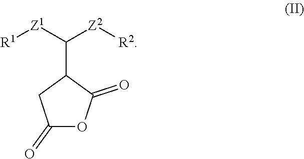

22. The process of clause 19, comprising
iii. contacting the compound of the formula II

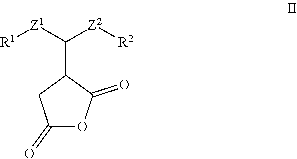

with an ammonia source under conditions sufficient to provide a compound of the formula I.

23. A process for preparing a compound of the formula I

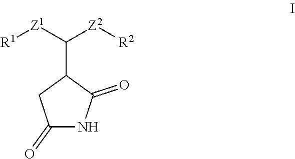

wherein
Z¹ and Z² are each independently a bond or —CH═CH—, provided that one of Z¹ or Z² is —CH═CH—, and one of Z¹ or Z² is a bond;
R¹ is H or C₁-C₆ alkyl; and
R² is C₆-C₃₆ alkyl,
comprising
i. contacting a terminal C₁₀-C₄₄ alkyl olefin with catalyst under conditions suitable to isomerize the terminal C₁₀-C₄₄ alkyl olefin to one or more of a beta-gamma C₁₀-C₄₄ alkyl olefin, gamma-delta C₁₀-C₄₄ alkyl olefin, delta-epsilon C₁₀-C₄₄ alkyl olefin, epsilon-zeta C₁₀-C₄₄ alkyl olefin, zeta-eta C₁₀-C₄₄ alkyl olefin, or eta-theta C₁₀-C₄₄ alkyl olefin; and
ii. contacting the beta-gamma C₁₀-C₄₄ alkyl olefin, gamma-delta C₁₀-C₄₄ alkyl olefin, delta-epsilon C₁₀-C₄₄ alkyl olefin, epsilon-zeta C₁₀-C₄₄ alkyl olefin, zeta-eta C₁₀-C₄₄ alkyl olefin, or eta-theta C₁₀-C₄₄ alkyl olefin product of step (i) with maleic anhydride under conditions suitable to provide a compound of the formula II

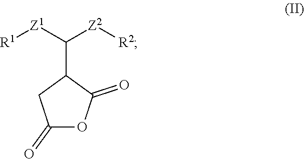

and
iii. contacting a compound of the formula II

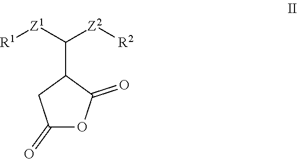

with an ammonia source under conditions sufficient to provide a compound of the formula I.

24. The process of any one of clauses 19, 20, or 23, wherein the catalyst used in step (i) is a solid acid catalyst, an aqueous acid catalyst, a supported metal catalyst, or an organometallic catalyst, or a combination of any one or more.

25. The process of any one of clauses 19, 20, 23, or 24, wherein the catalyst used in step (i) is sulfuric acid, p-toluenesulfonic acid, perfluorinated ion exchange resins (Nafion®), sulfonated poly(styrene-co-divinylbenzene) resins (PS-DVBs) including but not limited to Amberlyst 35, 70, XN1010, ZSM-35 and SAPO-11, tungstated zirconias, or acidic zeolites.

26. The process of any one of clauses 19, 20, 23 to 25, wherein the catalyst used in step (i) is Rhodium, Iridium, Nickel, Palladium, or Platinum.

27. The process of any one of clauses 19, 20, 23 to 26, wherein the catalyst used in step (i) is supported on carbon, aluminum oxide, titanium dioxide, silicon dioxide, zirconium dioxide, or magnesium oxide.

28. The process of any one of clauses 19, 20, or 23, wherein step (i) is carried out at a temperature of from about 25° C. to about 250° C.

29. The process of any one of clauses 19, 20, or 23, wherein the distribution of isomerized olefins in step (i) comprises one or more of a beta-gamma C₁₀-C₄₄ alkyl olefin, gamma-delta C₁₀-C₄₄ alkyl olefin, delta-epsilon C₁₀-C₄₄ alkyl olefin, epsilon-zeta C₁₀-C₄₄ alkyl olefin, or zeta-eta C₁₀-C₄₄ alkyl olefin.

30. The process of any one of clauses 19, 20, 23, or 29, wherein the distribution of isomerized olefins in step (i) comprises one or more of an alpha-beta C₁₀-C₄₄ alkyl olefin in an amount of from about 0 wt % to about 5 wt % of the distribution, beta-gamma C₁₀-C₄₄ alkyl olefin in an amount of from about 10 wt % to about 40 wt % of the distribution, gamma-delta C₁₀-C₄₄ alkyl olefin in an amount of from about 10 wt % to about 40 wt % of the distribution, delta-epsilon C₁₀-C₄₄ alkyl olefin in an amount of from about 10 wt % to about 40 wt % of the distribution, epsilon-zeta C₁₀-C₄₄ alkyl olefin in an amount of from about 10 wt % to about 40 wt % of the distribution, or zeta-eta C₁₀-C₄₄ alkyl olefin in an amount of from about 0 wt % to about 5 wt % of the distribution.

31. The process of any one of clauses 19, 20, 23, 29, or 30, wherein the distribution of isomerized olefins in step (i) comprises one or more of an alpha-beta C₁₀-C₄₄ alkyl olefin in an amount of from about 0 wt % to about 2 wt % of the distribution, beta-gamma C₁₀-C₄₄ alkyl olefin in an amount of from about 20 wt % to about 30 wt % of the distribution, gamma-delta C₁₀-C₄₄ alkyl olefin in an amount of from about 15 wt % to about 30 wt % of the distribution, delta-epsilon C₁₀-C₄₄alkyl olefin in an amount of from about 15 wt % to about 30 wt % of the distribution, epsilon-zeta C₁₀-C₄₄ alkyl olefin in an amount of from about 20 wt % to about 30 wt % of the distribution, or zeta-eta C₁₀-C₄₄ alkyl olefin in an amount of from about 0 wt % to about 2 wt % of the distribution.

32. The process of any one of clauses 19, 20, or 23, wherein one or more of the olefin in step (i) can be a C₁₀-C₄₄ terminal olefin.

33. The process of any one of clauses 19, 20, 23, or 32, wherein one or more of the olefin in step (i) can be a C₁₂-C₂₀ terminal olefin.

34. The process of any one of clauses 19, 20, 23, 32, or 33, wherein one or more of the olefin in step (i) can be a C₁₄-C₁₈ terminal olefin.

35. The process of any one of clauses 19, 20, 23, 32 to 34, wherein one or more of the olefin in step (i) can be a C₁₄ terminal olefin, or a C₁₆ terminal olefin, or a C₁₈ terminal olefin.

36. The process of any one of clauses 19, 21, or 23, wherein step (ii) is carried out at a temperature of from about 120° C. to about 250° C.

37. The process of any one of clauses 19, 21, 23, or 36, wherein step (ii) is carried out at a temperature of from about 200° C. to about 250° C.

38. The process of any one of clauses 19, 21, or 23, wherein step (ii) is carried out in a pressure reactor.

39. The process of any one of clauses 19, 21, 23, or 39 wherein step (ii) is carried out at a pressure of about 20 psi to 100 psi.

40. The process of any one of clauses 19, 21, 23, 39, or 40, wherein step (ii) is carried out at a pressure of about 40 psi to 80 psi.

41. The process of any one of clauses 19, 21, or 23, wherein step (ii) is carried out using the distribution of isomerized olefins in an amount equivalent to or in excess of the maleic anhydride.

42. The process of any one of clauses 19, 21, 23, or 41, wherein step (ii) is carried out using the distribution of isomerized olefins in an amount of from about 1.2 equivalents to about 2.0 equivalents relative to the maleic anhydride.

43. The process of any one of clauses 19, 21, or 23, wherein step (ii) is carried out from between about 1 hour to about 10 hours.

44. The process of any one of clauses 19, 21, 23, or 43, wherein step (ii) is carried out from between about 3 hours to about 7 hours.

45. The process of any one of clauses 19, 21, or 23, wherein step (ii) is carried out to cool the reaction vessel prior to filtration.

46. The process of any one of clauses 19, 21, 23, or 45, wherein step (ii) is carried out to cool the reaction vessel to a temperature of about 100° C. to about 200° C. prior to filtration.

47. The process of any one of clauses 19, 22, or 23, wherein step (iii) is carried out at a temperature of from about 120° C. to about 200° C.

48. The process of any one of clauses 19, 22, 23, or 47, wherein step (iii) is carried out at a temperature of from about 140° C. to about 180° C.

49. The process of any one of clauses 19, 22, 23, 47 or 48, wherein step (iii) is carried out in a pressure reactor.

50. The process of any one of clauses 19, 22, or 23, wherein step (iii) is carried out from between about 10 minutes to about 5 hours.

51. The process of any one of clauses 19, 22, 23, or 50, wherein step (iii) is carried out from between about 30 minutes to about 2 hours.

52. The process of any one of clauses 19 to 51, wherein the compound is of the formula Ia

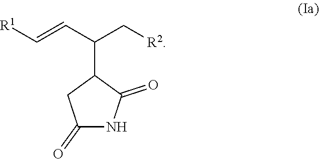

53. The process of any one of clauses 19 to 51, wherein the compound is of the formula Ib

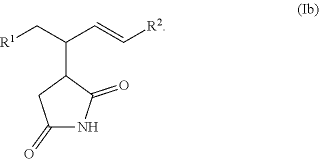

54. The process of any one of clauses 19 to 53, wherein R¹ is H or C₁-C₅ alkyl.

55. The process of any one of clauses 19 to 54, wherein R¹ is H, methyl, ethyl, n-propyl, n-butyl, or n-pentyl.

56. The process of any one of clauses 19 to 55, wherein R² is C₆-C₃₆ linear alkyl.

57. The process of any one of clauses 19 to 56, wherein R² is C₈-C₁₄ linear alkyl.

58. The process of any one of clauses 19 to 55, wherein R² is C₆-C₃₆ branched alkyl.

59. The process of any one of clauses 19 to 55, or 58, wherein R² is C₈-C₁₄ branched alkyl.

60. A compound of the formula I

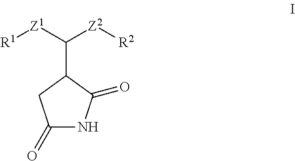

wherein
Z¹ and Z² are each independently a bond or —CH═CH—, provided that one of Z¹ or Z² is —CH═CH—, and one of Z¹ or Z² is a bond;
R¹ is H or C₁-C₆ alkyl; and
R² is C₆-C₃₆ alkyl,
wherein the compound of the formula I is prepared by a process according to any one of clauses 19 to 59.

61. A composition comprising at least one compound of the formula I

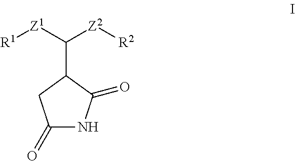

wherein
Z¹ and Z² are each independently a bond or —CH═CH—, provided that one of Z¹ or Z² is —CH═CH—, and one of Z¹ or Z² is a bond;
R¹ is H or C₁-C₆ alkyl; and
R² is C₆-C₃₆ alkyl,
wherein the compound of the formula I is prepared by a process according to any one of clauses 19 to 59.

### BRIEF DESCRIPTION OF THE DRAWINGS

FIG. 1 is a graph showing the lubricity performance results measuring wear scar diameter (WSD) by High frequency reciprocating rig (HFRR) testing using C16 succinimides according to ASTM D6079.

FIG. 2 is a graph showing the lubricity performance results measuring coefficient of friction (COF)) by High frequency reciprocating rig (HFRR) testing using C16 succinimides.

FIG. 3 is a picture showing the results of corrosion resistance testing using a mixture of C₁₆ succinimides tested according to ASTM 6594. Example 2 C16 succinimide mixture (left panel), control (right panel). Top to bottom both panels: Copper, Phosphor bronze, Tin, and Lead.

FIG. 4 is a graph showing the conductivity performance results measuring specific electrical conductivity over a temperature range using C16 succinimides according to DIN EN 51 111.

FIG. 5A shows the result of deposit test color rating using a baseline diesel fuel tested according to ASTM D3241.

FIG. 5B shows the result of deposit test color rating using a mixture of C16 succinimides tested according to ASTM D3241.

### DEFINITIONS

As used herein, the term “alkyl” includes a chain of carbon atoms, which is optionally branched and contains from 1 to 44 carbon atoms, or an alternate range, such as 1 to 12 carbons, or 1 to 20 carbons, and the like. Illustrative alkyl groups include, but are not limited to, methyl, ethyl, n-propyl, isopropyl, n-butyl, isobutyl, sec-butyl, tert-butyl, n-pentyl, hexyl, heptyl, octyl, nonyl, decyl, undecyl, dodecyl, tridecyl, tetradecyl, pentadecyl, hexadecyl, heptadecyl, octadecyl, nonadecyl, eicosyl, and the like. It will be appreciated that an alkyl group can be unsubstituted or substituted as described herein. An alkyl group can be substituted with any of the substituents in the various embodiments described herein, including one or more of such substituents.

As used herein, the term “alkenyl” or “alkenylene” includes a monovalent or divalent chain of carbon atoms, which is optionally branched, contains from 2 to 44 carbon atoms, or an alternate range, such as 2 to 12 carbons, or 2 to 20 carbons, and the like, and one or more carbon-carbon double bond (a.k.a. pi-bond). Illustrative alkenyl groups include, but are not limited to, vinyl, propenyl, isopropenyl, 1-butenyl, 2-butenyl, isobutenyl, 1-pentenyl, 2-pentenyl, 1-hexenyl, 2-hexenyl, 1-heptenyl, 2-heptenyl, 1-octenyl, 2-octenyl, 1-nonenyl, 2-nonenyl, 1-decenyl, 2-decenyl, 1-undecenyl, 1-dodecyl, 1-tridecenyl, 1-tetradecenyl, 1-pentadecenyl, 1-hexadecenyl, 1-heptadecenyl, 1-octadecenyl, 1-nonadecenyl, 1-eicosenyl, and the like. Illustrative alkenylene groups include, but are not limited to, vinylidene, propenylene, isopropenylene, 1-butenylene, 2-butenylene, isobutenylene, 1-pentenylene, 2-pentenylene, 1-hexenylene, 2-hexenylene, 1-heptenylene, 2-heptenylene, 1-octenylene, 2-octenylene, 1-nonenylene, 2-nonenylene, 1-decenylene, 2-decenylene, 1-undecenylene, 1-dodecylene, 1-tridecenylene, 1-tetradecenylene, 1-pentadecenylene, 1-hexadecenylene, 1-heptadecenylene, 1-octadecenylene, 1-nonadecenylene, 1-eicosenylene, and the like.

As used herein, the term “olefin” means a chain of carbon atoms, which is optionally branched, contains from 2 to 44 carbon atoms, or an alternate range, such as 2 to 12 carbons, or 2 to 20 carbons, and the like, and one or more carbon-carbon double bond (a.k.a. pi-bond). Illustrative alkyl olefins include, but are not limited to, ethene, propene, isopropene, 1-butene, 2-butene, isobutene, 1-pentene, 2-pentene, 1-hexene, 2-hexene, 1-heptene, 2-heptene, 1-octene, 2-octene, 1-nonene, 2-nonene, 1-decene, 2-decene, 1-undecene, 1-dodene, 1-tridecene, 1-tetradecene, 1-pentadecene, 1-hexadecene, 1-heptadecene, 1-octadecene, 1-nonadecene, 1-eicosene, and the like. Alternatively, the location of carbon-carbon double bond can be illustrated within the alkyl chain to include alpha-beta to describe a double bond between the first and second carbon of the alkyl chain, beta-gamma to describe a double bond between the second and third carbon of the alkyl chain, gamma-delta to describe a double bond between the third and fourth carbon of the alkyl chain, delta-epsilon to describe a double bond between the fourth and fifth carbon of the alkyl chain, epsilon-zeta to describe a double bond between the fifth and sixth carbon of the alkyl chain, zeta-eta to describe a double bond between the sixth and seventh carbon of the alkyl chain, and of the like. It will be appreciated that an alkenyl group or olefin can be unsubstituted or substituted as described herein. An alkyl olefin can be substituted with any of the substituents in the various embodiments described herein, including one or more of such substituents.

### DETAILED DESCRIPTION

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

As used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

### Representative Embodiments

Described herein is a carbon efficient approach to synthesize succinimide derivatives as described herein, such as those of the formula I, including but not limited to one or more hexadecenyl (C16 alkenyl) succinimide, and mixtures thereof. In some embodiments, the disclosure provides processes for preparing succinimide derivatives as described herein, such as those of the formula I, starting from an alpha alkyl olefin, such as hexadecene (C16 olefin). It will be appreciated that processes as described herein can provide a distribution of olefin derivatives useful in the preparation of anhydride derivatives as described herein by further transformation as described herein. According to the processes of the disclosure, anhydride derivatives as described herein can be readily converted to a succinimide derivatives as described herein, such as reacting with a nitrogen source additive, such as ammonia in presence of heat. In some embodiments, processes of the disclosure can be described according to Scheme 1.

### Step 1:

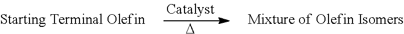

### Step 2

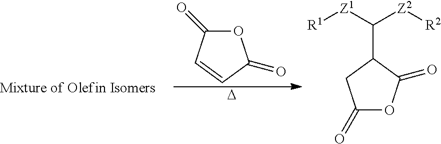

### Step 3:

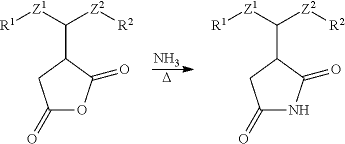

wherein R¹, R², Z¹, and Z² are as described herein.

It will be appreciated that the present disclosure provides processes for preparing a compound of the formula I described in the paragraphs above and below, comprising step (i) and one or more than one of the recited steps (ii) and (iii). Accordingly, the present disclosure provides a process for preparing a compound of the formula I, comprising step (i). Alternatively, the present disclosure provides a process for preparing a compound of the formula I, comprising steps (i) and (ii). Alternatively, the present disclosure provides a process for preparing a compound of the formula I, comprising steps (i), (ii), and (iii). Alternatively, the present disclosure provides a process for preparing a compound of the formula II, comprising step (ii). Alternatively, the present disclosure provides a process for preparing a compound of the formula II, comprising steps (i) and (ii).

In step (i), a terminal C₁₀-C₄₄ alkyl olefin with a catalyst under conditions suitable to isomerize the terminal C₁₀-C₄₄ alkyl olefin to one or more of a beta-gamma C₁₀-C₄₄ alkyl olefin, gamma-delta C₁₀-C₄₄ alkyl olefin, delta-epsilon C₁₀-C₄₄ alkyl olefin, epsilon-zeta C₁₀-C₄₄ alkyl olefin, zeta-eta C₁₀-C₄₄ alkyl olefin, or eta-theta C₁₀-C₄₄ alkyl olefin.

In step (i), methods for the isomerization of terminal olefins are known in the art. The skilled person will recognize that a variety of methods known in the art can be applied to the isomerization step (i) described herein. Examples of suitable conditions and catalysts useful in the isomerization of terminal olefins can be found in, for example, Huss, A. Jr., EP0765300B1 (Feb. 9, 2000); Capwell, David A. US2006/0100474A1 (May 11, 2006); Ramachandran, B., US2015/0141720A1 (May 21, 2015); Bruno, J. E., “Catalysts for the Positional Isomerization of Internal, Long-Chain Olefins,” LSU Doctoral Dissertations, 2015; Fiorito, D., “Transition metal-catalyzed alkene isomerization as an enabling technology in tandem, sequential and domino processes,” Chem. Soc. Rev., 2021; Ma, W., “Catalytic Isomerization of Olefins and Their Derivatives: A Brief Overview,” Alkenes, Aug. 8, 2021, the disclosures of each of which, as it pertains to methods, conditions, and catalysts for the isomerization of terminal olefins, is incorporated herein by reference.”

In step (i), the catalyst can be any suitable catalyst or a combination of any one or more such catalysts. Suitable catalysts include, but are not limited to, a solid acid catalyst, an aqueous acid catalyst, a supported metal catalyst, or an organometallic catalyst, and combinations thereof. In some embodiments, the suitable catalyst can include, but is not limited to, sulfuric acid, p-toluenesulfonic acid, perfluorinated ion exchange resins (Nafion®), sulfonated poly(styrene-co-divinylbenzene) resins (PS-DVBs), including but not limited to Amberlyst 35, 70, XN1010, ZSM-35 and SAPO-11, tungstated zirconias, or acidic zeolites. In some embodiments, suitable catalysts can include, but are not limited to, rhodium, iridium, nickel, palladium, or platinum catalysts. In some embodiments, suitable catalysts can include, but are not limited to, metal catalysts supported on carbon (C), aluminum oxide (Al₂O₃), titanium dioxide (TiO₂), silicon dioxide (SiO₂), zirconium dioxide (ZrO₂), or magnesium oxide (MgO). It will be appreciated that any supported metal catalyst known in the art for olefin isomerization can be applied to the present disclosure to obtain an isomerized product or isomerized product mixture with a desired purity or product mixture distribution. It will further be appreciated that any combination of metals and supports mentioned above known in the art, such as palladium on carbon (Pd/C) or nickel on silica can be used in the methods described herein.

It will be appreciated that step (i) can be conducted at any temperature commonly used in connection with olefin isomerization chemistry processes, such as room temperature or under warming conditions. In some embodiments, step (i) can be carried out at a temperature of about 25° C. to about 250° C.

In some embodiments of step (i), the distribution of isomerized olefins comprises one or more of a beta-gamma C₁₀-C₄₄ alkyl olefin, gamma-delta C₁₀-C₄₄ alkyl olefin, delta-epsilon C₁₀-C₄₄ alkyl olefin, epsilon-zeta C₁₀-C₄₄ alkyl olefin, or zeta-eta C₁₀-C₄₄ alkyl olefin. In some embodiments of step (i), the distribution of isomerized olefins comprises one or more of an alpha-beta C₁₀-C₄₄ alkyl olefin in an amount of from about 0 wt % to about 5 wt % of the distribution, beta-gamma C₁₀-C₄₄ alkyl olefin in an amount of from about 10 wt % to about 40 wt % of the distribution, gamma-delta C₁₀-C₄₄ alkyl olefin in an amount of from about 10 wt % to about 40 wt % of the distribution, delta-epsilon C₁-C₄₄ alkyl olefin in an amount of from about 10 wt % to about 40 wt % of the distribution, epsilon-zeta C₁₀-C₄₄ alkyl olefin in an amount of from about 10 wt % to about 40 wt % of the distribution, or zeta-eta C₁₀-C₄₄ alkyl olefin in an amount of from about 0 wt % to about 5 wt % of the distribution. In some embodiments of step (i), the distribution of isomerized olefins comprises one or more of an alpha-beta C₁₀-C₄₄ alkyl olefin in an amount of from about 0 wt % to about 2 wt % of the distribution, beta-gamma C₁₀-C₄₄ alkyl olefin in an amount of from about 20 wt % to about 30 wt % of the distribution, gamma-delta C₁₀-C₄₄ alkyl olefin in an amount of from about 15 wt % to about 30 wt % of the distribution, delta-epsilon C₁₀-C₄₄ alkyl olefin in an amount of from about 15 wt % to about 30 wt % of the distribution, epsilon-zeta C₁₀-C₄₄ alkyl olefin in an amount of from about 20 wt % to about 30 wt % of the distribution, or zeta-eta C₁₀-C₄₄ alkyl olefin in an amount of from about 0 wt % to about 2 wt % of the distribution.

In some embodiments of step (i), the terminal olefin can be a C₁₀-C₄₄ terminal olefin. In some embodiments of step (i), the terminal olefin can be a C₁₂-C₂₀ terminal olefin. In some embodiments of step (i), the terminal olefin can be a C₁₄-C₁₅ terminal olefin. In some embodiments of step (i), the terminal olefin can be a C₁₄ terminal olefin, or a C₁₆ terminal olefin, or a C₁₈ terminal olefin.

In step (ii), the anhydride can be any suitable anhydride, such as maleic anhydride. It will be appreciated that step (ii) can be conducted at any temperature commonly used in connection with maleic anhydride coupling chemistry processes, such as under warming conditions or under pressure. In some embodiments, step (ii) can be carried out at a temperature of about 120° C. to about 250° C. In some embodiments, step (ii) can be carried out at a temperature of about 200° C. to about 250° C. In some embodiments, step (ii) can be carried out at a pressure of about 20 psi to 100 psi. In some embodiments, step (ii) can be carried out at a pressure of about 40 psi to 80 psi. In some embodiments, step (ii) can be carried out using the distribution of isomerized olefins in an amount equivalent to or in excess of the maleic anhydride. In some embodiments, step (ii) can be carried out using the distribution of isomerized olefins in an amount of from about 1.2 equivalents to about 2.0 equivalents relative to the maleic anhydride. In some embodiments, step (ii) can be carried out from between about 1 hour to about 10 hours. In some embodiments, step (ii) can be carried out from between about 3 hour to about 7 hours. In some embodiments, it may be advantageous in step (ii) to cool the reaction vessel prior to filtration. In some embodiments, it may be advantageous in step (ii) to cool the reaction vessel to a temperature of about 100° C. to about 200° C. prior to filtration.

In some embodiments of step (ii), the compound of the formula II can be of the formula

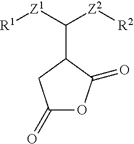

wherein
Z¹ and Z² are each independently a bond or —CH═CH—, provided that one of Z¹ or Z² is —CH═CH—, and one of Z¹ or Z² is a bond;
R¹ is H or C₁-C₆ alkyl; and
R² is C₆-C₃₆ alkyl.

In some embodiments of step (ii), the compound of the formula II is selected from the group consisting of

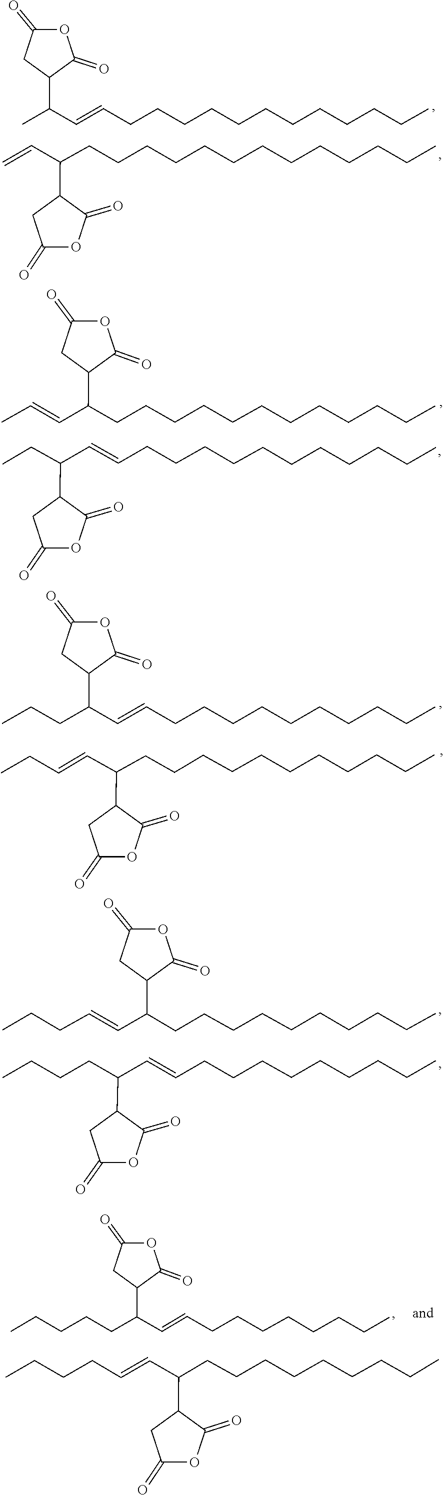

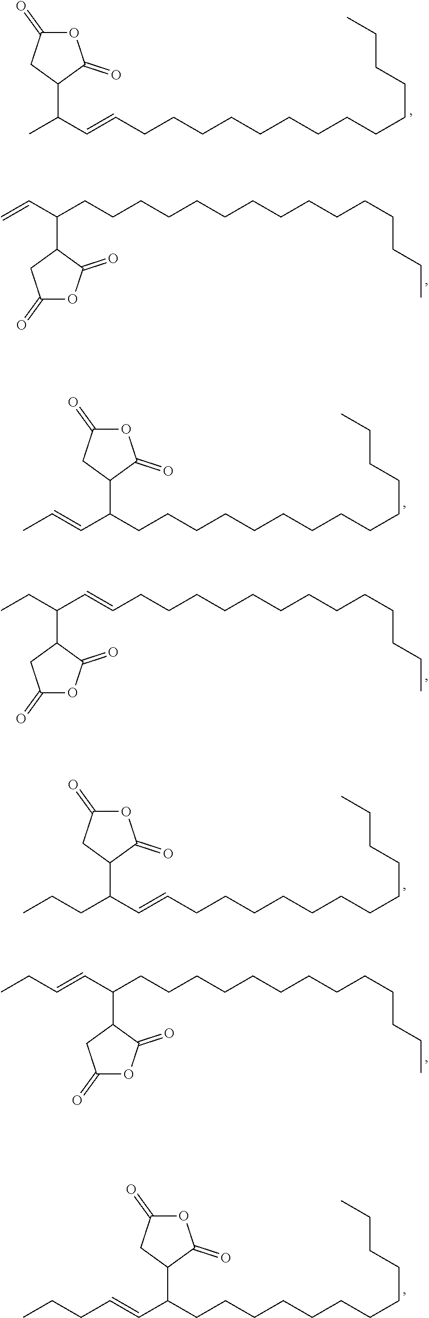

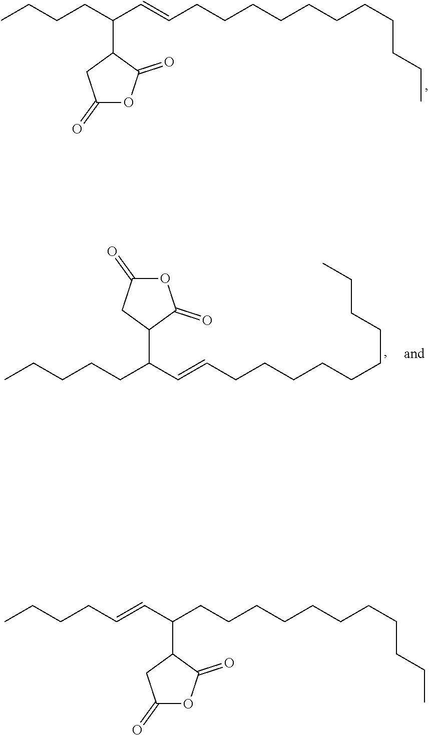

In step (iii), the nitrogen source can be any suitable nitrogen source additive, such as ammonia. It will be appreciated that step (iii) can be conducted at any temperature commonly used in connection with succinimide conversion chemistry processes, such as under warming conditions. In some embodiments, step (iii) can be carried out at a temperature of about 120° C. to about 200° C. In some embodiments, step (iii) can be carried out at a temperature of about 140° C. to about 180° C. In some embodiments, step (ii) can be carried out from between about 10 minutes to about 5 hours. In some embodiments, step (ii) can be carried out from between about 30 minutes to about 2 hours.

In step (iii), the compound of the formula I can be of the formula

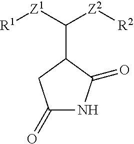

wherein
Z¹ and Z² are each independently a bond or —CH═CH—, provided that one of Z¹ or Z² is —CH═CH—, and one of Z¹ or Z² is a bond;
R¹ is H or C₁-C₆ alkyl; and
R² is C₆-C₃₆ alkyl.

In some embodiments, the compound of the formula I has an electrical conductivity of less than about 100 nS/m. In some embodiments, the compound of the formula I has an electrical conductivity of less than about 90 nS/m. In some embodiments, the compound of the formula I has an electrical conductivity of less than about 80 nS/m. In some embodiments, the compound of the formula I has an electrical conductivity of less than about 70 nS/m. In some embodiments, the compound of the formula I has an electrical conductivity of less than about 100 nS/m when measured over a range of from about 20° C. to about 160° C. In some embodiments, the compound of the formula I has an electrical conductivity of less than about 90 nS/m when measured over a range of from about 20° C. to about 160° C. In some embodiments, the compound of the formula I has an electrical conductivity of less than about 80 nS/m when measured over a range of from about 20° C. to about 160° C. In some embodiments, the compound of the formula I has an electrical conductivity of less than about 70 nS/m when measured over a range of from about 20° C. to about 160° C. In some embodiments, the compound of the formula I has an electrical conductivity of less than about 70 nS/m when measured over a range of from about 20° C. to about 150° C. In some embodiments, the compound of the formula I has an electrical conductivity of less than about 50 nS/m when measured over a range of from about 20° C. to about 140° C. In some embodiments, the compound of the formula I has an electrical conductivity of less than about 20 nS/m when measured over a range of from about 20° C. to about 120° C. In some embodiments, the compound of the formula I has an electrical conductivity of less than about 10 nS/m when measured over a range of from about 20° C. to about 120° C. In some embodiments, the compound of the formula I has an electrical conductivity of less than about 5 nS/m when measured over a range of from about 20° C. to about 110° C. In some embodiments, the compound of the formula I has an electrical conductivity of less than about 2 nS/m when measured over a range of from about 20° C. to about 100° C.

In some embodiments, the compound of the formula I has an electrical conductivity in a range values between about 0 nS/m and about 2 nS/m when measured over a range of from about 20° C. to about 70° C. In some embodiments, the compound of the formula I has an electrical conductivity in a range values between about 2 nS/m and about 5 nS/m when measured over a range of from about 80° C. to about 90° C. In some embodiments, the compound of the formula I has an electrical conductivity in a range values between about 2 nS/m and about 10 nS/m when measured over a range of from about 80° C. to about 100° C. In some embodiments, the compound of the formula I has an electrical conductivity in a range values between about 5 nS/m and about 25 nS/m when measured over a range of from about 100° C. to about 120° C. In some embodiments, the compound of the formula I has an electrical conductivity in a range values between about 20 nS/m and about 70 nS/m when measured over a range of from about 120° C. to about 150° C. In some embodiments, the compound of the formula I has an electrical conductivity in a range values between about 5 nS/m and about 70 nS/m when measured over a range of from about 100° C. to about 150° C. In some embodiments, the compound of the formula I has an electrical conductivity in a range values between about 2 nS/m and about 70 nS/m when measured over a range of from about 80° C. to about 150° C. It will be appreciated that the electrical conductivity of the compound of the formula I can be measured using the DIN EN 51 111 test standard.

In some embodiments, the compound of the formula I has dielectric breakdown voltage of greater than about 30 kV when tested under ASTM D1816 (1 mm). In some embodiments, the compound of the formula I has an average dielectric breakdown voltage of greater than about 45 kV when tested under ASTM D1816 (1 mm). In some embodiments, the compound of the formula I has dielectric breakdown voltage in the range of about 30 kV to 55 kV when tested under ASTM D1816 (1 mm). In some embodiments, the compound of the formula I has dielectric breakdown voltage of greater than about 55 kV when tested under ASTM D1816 (2 mm). In some embodiments, the compound of the formula I has an average dielectric breakdown voltage of greater than about 70 kV when tested under ASTM D1816 (2 mm). In some embodiments, the compound of the formula I has dielectric breakdown voltage in the range of about 57 kV to 74 kV when tested under ASTM D1816 (2 mm). In some embodiments, the compound of the formula I dielectric breakdown voltage can be measured at a temperature of from about 20° C. to about 25° C. according to ASTM D1816 (1 mm) or ASTM D1816 (2 mm).

In some embodiments of step (iii), the compound of the formula I is selected from the group consisting of

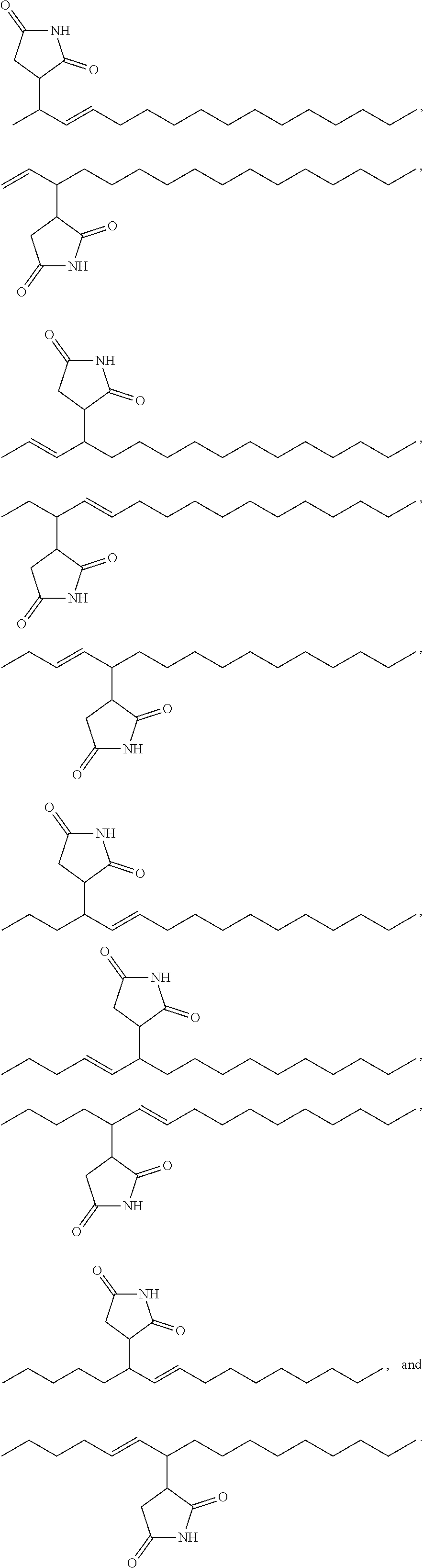

In some embodiments of step (iii), the compound of the formula I is selected from the group consisting of

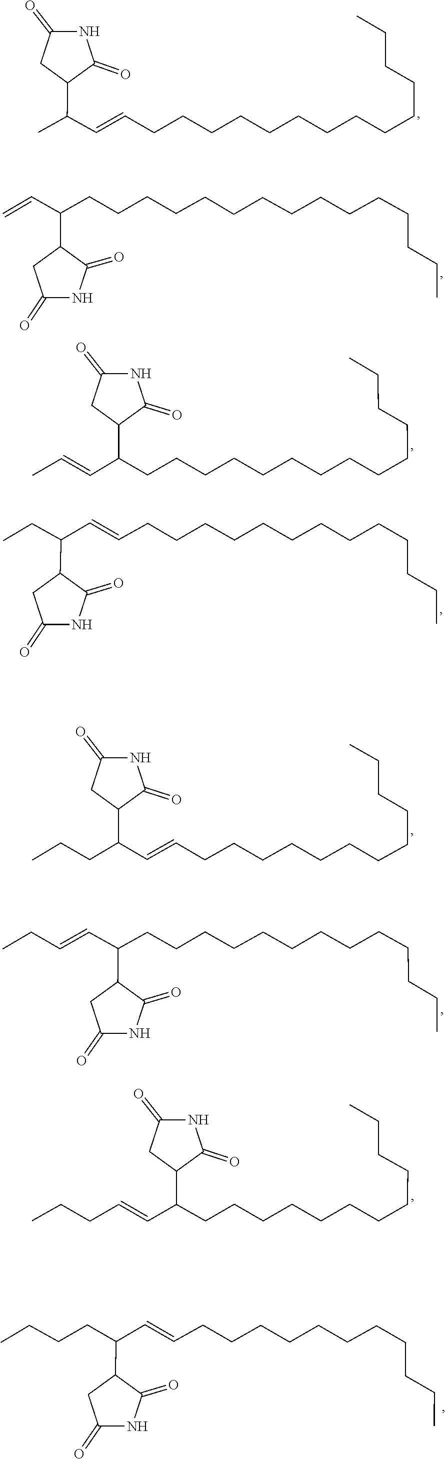

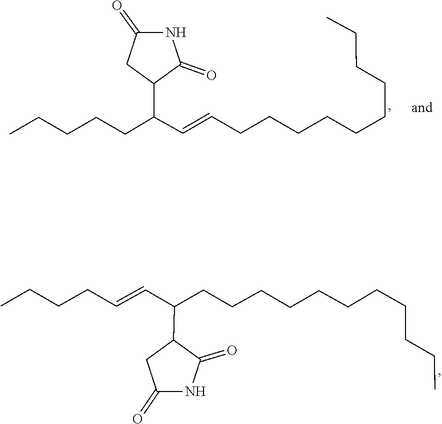 [SMILES: CCCCCCCCCC/C=C/C(CCCCC)C1CC(=O)NC1=O ; CCCC/C=C/C(CCCCCCCCCCC)C1CC(=O)NC1=O]

In some embodiments, the disclosure provides a composition comprising at least one compound of the formula I and one or more of an oil (such as a diesel oil), a fuel (such as gasoline or diesel), and the like. In some embodiments, the composition comprising at least one compound of the formula I and one or more of an oil (such as a diesel oil), a fuel (such as gasoline or diesel), and the like has a wear scar diameter of less than about 520 μm. In some embodiments, the composition comprising at least one compound of the formula I and one or more of an oil (such as a diesel oil), a fuel (such as gasoline or diesel), and the like has a wear scar diameter of less than about 460 μm. In some embodiments, the composition comprising at least one compound of the formula I and one or more of an oil (such as a diesel oil), a fuel (such as gasoline or diesel), and the like has a wear scar diameter of less than about 400 μm. In some embodiments, the composition comprising at least one compound of the formula I and one or more of an oil (such as a diesel oil), a fuel (such as gasoline or diesel), and the like has a wear scar diameter of less than about 350 μm.

In some embodiments, the composition comprising at least one compound of the formula I and one or more of an oil (such as a diesel oil), a fuel (such as gasoline or diesel), and the like has a wear scar diameter of less than about 520 μm at a dosage level of the at least one compound of the formula I that is greater than about 50 ppmv in the composition. In some embodiments, the composition comprising at least one compound of the formula I and one or more of an oil (such as a diesel oil), a fuel (such as gasoline or diesel), and the like has a wear scar diameter of less than about 520 μm at a dosage level of the at least one compound of the formula I that is greater than about 75 ppmv in the composition. In some embodiments, the composition comprising at least one compound of the formula I and one or more of an oil (such as a diesel oil), a fuel (such as gasoline or diesel), and the like has a wear scar diameter of less than about 460 μm at a dosage level of the at least one compound of the formula I that is greater than about 100 ppmv in the composition. In some embodiments, the composition comprising at least one compound of the formula I and one or more of an oil (such as a diesel oil), a fuel (such as gasoline or diesel), and the like has a wear scar diameter of less than about 460 μm at a dosage level of the at least one compound of the formula I that is greater than about 110 ppmv in the composition. In some embodiments, the composition comprising at least one compound of the formula I and one or more of an oil (such as a diesel oil), a fuel (such as gasoline or diesel), and the like has a wear scar diameter of less than about 460 μm at a dosage level of the at least one compound of the formula I that is greater than about 120 ppmv in the composition. In some embodiments, the composition comprising at least one compound of the formula I and one or more of an oil (such as a diesel oil), a fuel (such as gasoline or diesel), and the like has a wear scar diameter of less than about 400 μm at a dosage level of the at least one compound of the formula I that is greater than about 200 ppmv in the composition. In some embodiments, the composition comprising at least one compound of the formula I and one or more of an oil (such as a diesel oil), a fuel (such as gasoline or diesel), and the like has a wear scar diameter of less than about 350 μm at a dosage level of the at least one compound of the formula I that is greater than about 300 ppmv in the composition. It will be appreciated that the lubricity performance can be measured by any one of several standard tests to quantify the suitability of fuels for lubricity requirements, such as CEC F-06-A-96, ASTM D6079, ASTM 7688, ISO BS EN 590, ISO 12156-1, JPI-5S-50-98 and IP 450/2000

In some embodiments, the disclosure provides an electric transmission fluid, an electric thermal fluid, or an electric grease comprising at least one compound of the formula T.

### EXAMPLES

The examples and preparations provided below further illustrate and exemplify particular aspects of embodiments of the disclosure. It is to be understood that the scope of the present disclosure is not limited in any way by the scope of the following examples.

### Example 1

907 grams (4.04 moles) of isomerized hexadecene (made according to the methods described herein) was placed in a stainless steel vessel capable of withstanding a pressure of at least 50 psi and capable of agitation and capable of heating. The vessel was heated to 230° C. with agitation. 270 grams (2.69 moles) of maleic anhydride was melted and added to the reactor. The reaction was ran at 230° C. for 5 hours. At the end of this period, the excess maleic anhydride and isomerized hexadecene was removed with vacuum. The reactor was cooled to 150° C. The product was removed and filtered with a 20 μm filter. A yield of 732 grams of product was obtained.

### Example 2

732 grams (2.27 moles) of product from Example 1 was added to the same vessel as Example 1. The vessel was heated to 160° C. The vessel was sparged with ammonia through a dip tube at 160° C. for 1 hour while open to a receiving vessel that collects the water produced during the reaction. The excess dissolved ammonia and water was removed with vacuum. A yield of 695 grams of product was obtained.

### Example 3

High frequency reciprocating rig (HFRR) is an industry recognized test for measuring the lubricity of fuels and lubricants. Due to the widespread introduction of low sulfur fuel, inherent lubricity of fuel is significantly reduced, therefore compromising wear and deposit control. Lubricity is a critical parameter of fuel quality and is important to maintain proper functioning of the fuel injection system hardware. Industry has adopted the following standard tests to quantify the suitability of fuels for lubricity requirements: CEC F-06-A-96, ASTM D6079, ASTM 7688, ISO BS EN 590, ISO 12156-1, JPI-5S-50-98 and IP 450/2000. The test procedure involves high frequency (50 Hz) repeated rubbing of a 5 mm alloy steel ball on a 10 mm alloy steel disk under 1.96N load in the presence of a fuel sample maintained at 60° C. for 75 minutes. Experiments were run with clay treated #2 diesel fuel (baseline) with and without product prepared by Example 2 added at a specific concentration (ppmv) in baseline diesel fuel. Wear scar diameter (WSD) on the ball was measured in both parallel and perpendicular to the sliding direction, and the average wear scar was reported. The WSD limit in the US is 520 μm, in Europe 460 μm and Worldwide Fuel Charter requires a limit of 400 μm.

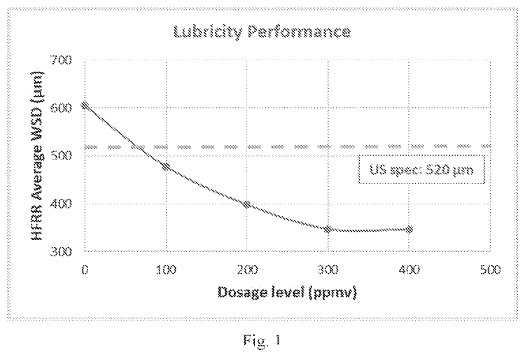

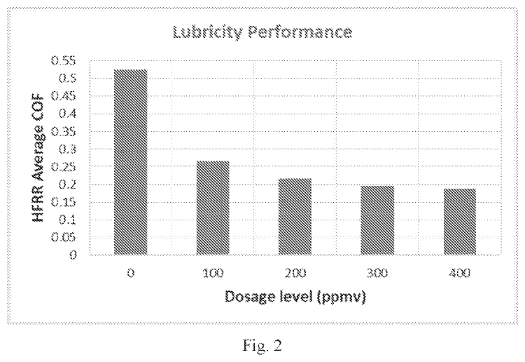

Lubricity performance was measured using the mixture of C16 succinimides produced by Example 2 according to ASTM D6079. The results are shown in Table 1, Table 2, and FIG. 1. Coefficient of friction test results are shown in Table 3 and FIG. 2.

TABLE 1

| | | C16 succinimide | | |
|---|---|---|---|---|
| | No | from Ex. 2 | Industry | Industry |
| | Additive | (100 ppmv | Maxi- | Pre- |
| | Diesel | active) | mum | ferred |
| Wear Scar Run 1 (μm) | 615 | 441 | | |
| Wear Scar Run 2 (μm) | 580 | 436 | | |
| Wear Scar Run 3 (μm) | 581 | 454 | | |
| Wear Scar Average | 592 | 444 | 520 | 460 |
| (μm) | | | | |

TABLE 2

| | | C16 | C16 | C16 | C16 | | |
|---|---|---|---|---|---|---|---|
| | No | succinimide | succinimide | succinimide | succinimide | | |
| | Additive | (100 ppmv | (200 ppmv | (300 ppmv | (400 ppmv | Industry | Industry |
| | Diesel | active) | active) | active) | active) | Maximum | Preferred |
| Wear | 602 | 477 | 387 | 347 | 350 | | |
| Scar | | | | | | | |
| Run 1 | | | | | | | |
| (μm) | | | | | | | |
| Wear | 608 | 478 | 409 | 347 | 342 | | |
| Scar | | | | | | | |
| Run 2 | | | | | | | |
| (μm) | | | | | | | |
| Wear | 605 | 478 | 398 | 347 | 346 | 520 | 460 |
| Scar | | | | | | | |
| Average | | | | | | | |
| (μm) | | | | | | | |

TABLE 3

| | | C16 | C16 | C16 | C16 |
|---|---|---|---|---|---|
| | No | succin- | succin- | succin | succin- |
| | Addi- | imide | imide | imide | imide |
| | tive | (100 ppmv | (200 ppmv | (300 ppmv | (400 ppmv |
| | Diesel | active) | active) | active) | active) |
| Coefficient of | 0.508 | 0.270 | 0.217 | 0.195 | 0.187 |
| Friction Run 1 | | | | | |
| Coefficient of | 0.544 | 0.264 | 0.217 | 0.196 | 0.189 |
| Friction Run 1 | | | | | |
| Average | 0.526 | 0.267 | 0.217 | 0.196 | 0.188 |
| Coefficient of | | | | | |
| Friction | | | | | |

### Example 4

Corrosion is a chemical process where the metal undergoes oxidation with the gradual degradation and deterioration of the metal surface. It is a common failure mode observed with fuel and lubricant applications and can be prevented by using suitable chemistry to passivate the metal surface. ASTM D6594 was adopted to test the corrosive property of the test sample. The test was carried out with a baseline (blank fluid) and Example 2 (1000 ppm by weight) doped baseline fluid at 135° C. for lead corrosion. After the completion of test, lead loss was measured in the end of test (EOT) fluid in ppm using Inductively Coupled Plasma Atomic Emission Spectrometric (ICP-AES) method.

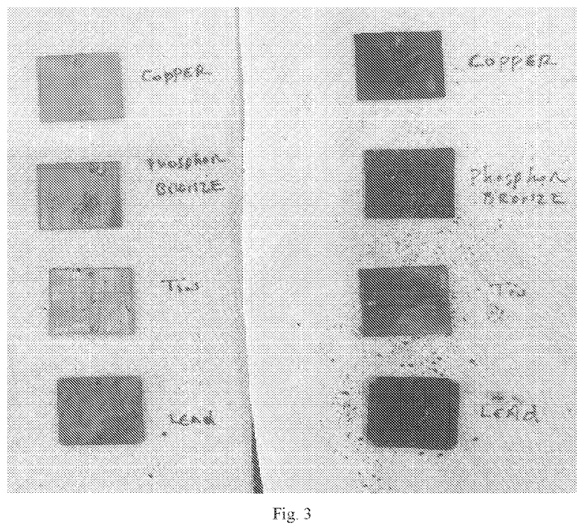

Corrosion Performance was measured using the mixture of C16 succinimides produced by Example 2 according to ASTM D6594. The results are shown in Table 4 (ICP Analysis of Leached Lead in Test Fluid) and FIG. 3.

TABLE 4

| Sample ID | Leached Lead (ppm) |
|---|---|
| Blank Fluid | 739 |
| Used Fluid With Ex. 2 C16 | 659 |

### Example 5

Electrical conductivity is one of the key parameters to determine the suitability of lubricant used as electric drive fluid (EDF). Lubricants used in eMotor are designed to deliver appropriate low conductivity to prevent charge build-up and prevent arcing and cause hardware damage. External factors such as temperature, time and moisture content can alter fluid conductivity. Polar components arising from oxidation will also increase the ability of the lubricant to carry charge. For optimal performance, EDF is expected to deliver low and stable electrical conductivity over the operational life of the lubricant. The conductivity measurements were run using Epsilon+Dielectricity Meter according to the requirements of DIN EN 51 111 test standard. Measurement of the specific electrical conductivity of Example 2 was run in neat condition with applied frequency 20 Hz over a broad temperature range of 20 to 150° C.

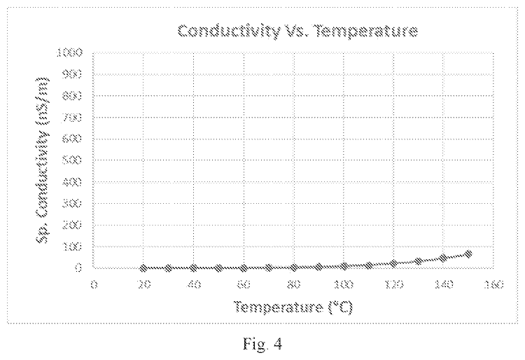

Conductivity performance was measured using the mixture of C16 succinimides produced by Example 2 according to DIN EN 51 111 test standard. The results are shown in Table 5 and FIG. 4.

TABLE 5

| | spec. |
|---|---|
| Temperature | Conductance |
| [° C.] | [nS/m] |
| 20.2 | 0.015 |
| 30.0 | 0.045 |
| 40.0 | 0.134 |
| 50.0 | 0.333 |
| 60.0 | 0.743 |
| 70.0 | 1.510 |
| 80.0 | 2.827 |
| 90.0 | 4.986 |
| 100.0 | 8.336 |
| 110.0 | 13.387 |
| 120.0 | 20.953 |
| 130.0 | 31.611 |
| 140.0 | 46.007 |
| 150.0 | 64.625 |

### Example 6

Dielectric breakdown voltage is a measure of the resistance of an insulator to break down under an applied electric field. This breakdown of the insulation often results in arcing which can lead to hardware failure in high voltage applications. Breakdown voltages of Example 2 were measured in neat condition according to ASTM D1816-12 test method in 1- and 2-mm test cell at room temperature (22.6° C.) and humidity 38.6%. The tests were repeated 5 times and between individual breakdowns, the test sample was stirred for 1 minute.

Breakdown voltage was measured using the mixture of C16 succinimides produced by Example 2 according to ASTM D1816. The results are shown in Table 6.

TABLE 6

| Test | ASTM D1816 (1 mm) | ASTM D1816 (2 mm) |
|---|---|---|
| Measurement 1 | 30.3 | >73.7 |
| Measurement 2 | 47.8 | 57.6 |
| Measurement 3 | 47.1 | 71.2 |
| Measurement 4 | 51.3 | >73.7 |
| Measurement 5 | 50.1 | >73.7 |
| Average | 45.3 | 70.0 |

### Example 7

Oxidative degradation of fuels and oils results in insoluble and soluble materials that are the precursors of engine deposit and lacquers often observed in high temperature applications. ASTM test D3241 was adopted to determine deposit forming tendency of clay treated #2 diesel fuel with and without the presence of deposit inhibiting chemistry. The test is carried out by passing test fuel sample over a heated aluminum alloy tube at a constant flow rate. At the end of the test the tube is removed and inspected for any deposit, stain, or discoloration and rated comparing it to a standard color chart. The method simulates conditions for fuel degradation as observed in automotive fuel injection system. The observed rating of baseline test is an indication of fuel oxidation, while the product rating illustrates the efficiency of Example 2 chemistry to prevent formation of insoluble deposit on a heated metal surface at a desired test temperature.

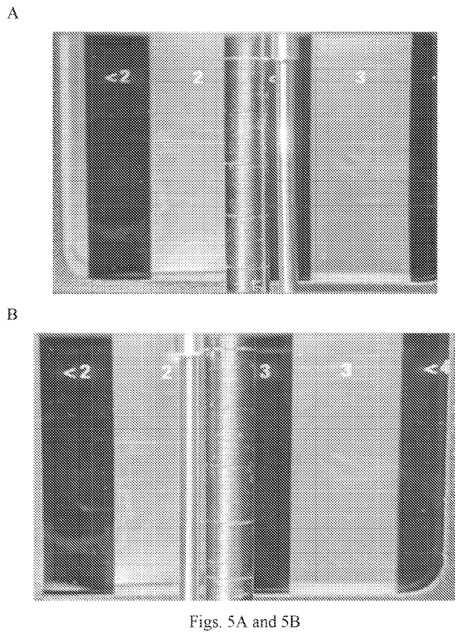

Simulated oxidation was measured using the mixture of C16 succinimides produced by Example 2 according to ASTM 3241. The results of deposit test color rating are shown in Table 7 and FIGS. 5A and 5B.

TABLE 7

| Sample | Description | Oxidation rating |
|---|---|---|
| Baseline | Clay treated #2 diesel fuel | <3 |
| Product | 800 μL blended in 4000 ml | 2 |
| | clay treated #2 diesel fuel | |

1. A compound of the formula I 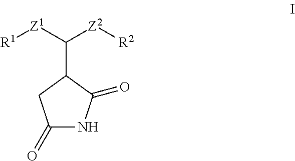
wherein
Z¹ and Z² are each independently a bond or —CH═CH—, provided that one of Z¹ or Z² is —CH═CH—, and one of Z¹ or Z² is a bond;
R¹ is H or C₁-C₆ alkyl; and
R² is C₆-C₃₆ alkyl.

2. The compound of claim 1, wherein the compound is of the formula Ia

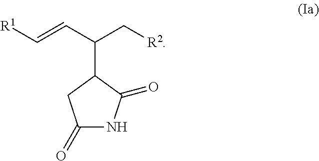

3. The compound of claim 1, wherein the compound is of the formula Ib

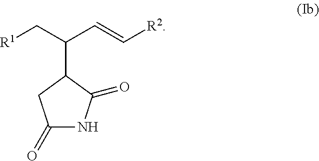

4. The compound of claim 1, wherein R¹ is H or C₁-C₅ alkyl.

5. The compound of claim 1, wherein R¹ is H, methyl, ethyl, n-propyl, n-butyl, or n-pentyl.

6. The compound of claim 5, wherein R² is C₆-C₃₆ linear alkyl.

7. The compound of claim 5, wherein R² is C₈-C₁₄ linear alkyl.

8. The compound of claim 5, wherein R² is C₆-C₃₆ branched alkyl.

9. The compound of claim 5, wherein R² is C₈-C₁₄ branched alkyl.

10-18. (canceled)

19. A process for preparing a compound of the formula I 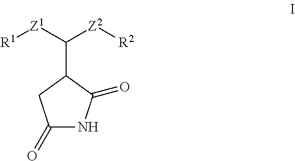
wherein
Z¹ and Z² are each independently a bond or —CH═CH—, provided that one of Z¹ or Z² is —CH═CH—, and one of Z¹ or Z² is a bond;
R¹ is H or C₁-C₆ alkyl; and
R² is C₆-C₃₆ alkyl,
comprising one or more of steps
i. contacting a terminal C₁₀-C₄₄ alkyl olefin with a catalyst under conditions suitable to isomerize the terminal C₁₀-C₄₄ alkyl olefin to one or more of a beta-gamma Cia-C₄₄ alkyl olefin, gamma-delta C₁₀-C₄₄ alkyl olefin, delta-epsilon C₁₀-C₄₄ alkyl olefin, epsilon-zeta C₁₀-C₄₄ alkyl olefin, or zeta-eta C₁₀-C₄₄ alkyl olefin; or
ii. contacting the beta-gamma C₁₀-C₄₄ alkyl olefin, gamma-delta C₁₀-C₄₄ alkyl olefin, delta-epsilon C₁₀-C₄₄ alkyl olefin, epsilon-zeta C₁₀-C₄₄ alkyl olefin, or zeta-eta C₁₁-C₄₄ alkyl olefin product of step (i) with maleic anhydride under conditions suitable to provide a compound of the formula II 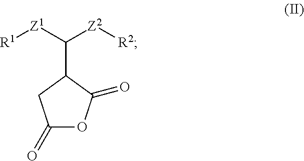
or
iii. contacting a compound of the formula II 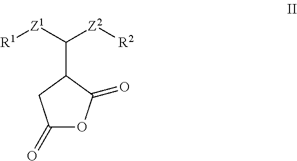
with an ammonia source under conditions sufficient to provide a compound of the formula I.

20. The process of claim 19, comprising
i. contacting a terminal C₁₀-C₄₄ alkyl olefin with a catalyst under conditions suitable to isomerize the terminal C₁₀-C₄₄ alkyl olefin to one or more of a beta-gamma C₁₀-C₄₄ alkyl olefin, gamma-delta C₁₀-C₄₄ alkyl olefin, delta-epsilon C₁₀-C₄₄ alkyl olefin, epsilon-zeta C₁₀-C₄₄ alkyl olefin, or zeta-eta C₁₀-C₄₄ alkyl olefin.

21. The process of claim 19, comprising
ii. contacting a beta-gamma beta-gamma C₁₀-C₄₄ alkyl olefin, gamma-delta C₁₀-C₄₄ alkyl olefin, delta-epsilon C₁₀-C₄₄ alkyl olefin, epsilon-zeta C₁₀-C₄₄ alkyl olefin, or zeta-eta C₁₀-C₄₄ alkyl olefin product of step (i) with maleic anhydride under conditions suitable to provide a compound of the formula II

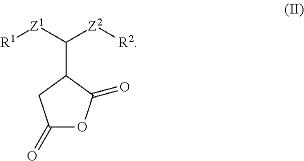

22. The process of claim 19, comprising
iii. contacting the compound of the formula II 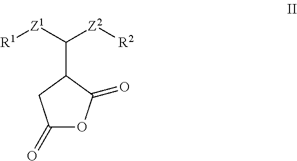
with an ammonia source under conditions sufficient to provide a compound of the formula I.

23. A process for preparing a compound of the formula I 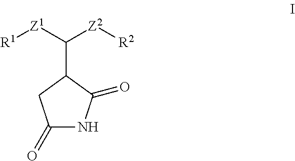
wherein
Z¹ and Z² are each independently a bond or —CH═CH—, provided that one of Z¹ or Z² is —CH═CH—, and one of Z¹ or Z² is a bond;
R¹ is H or C₁-C₆ alkyl; and
R² is C₆-C₃₆ alkyl,
comprising
i. contacting a terminal C₁₀-C₄₄ alkyl olefin with catalyst under conditions suitable to isomerize the terminal C₁₀-C₄₄ alkyl olefin to one or more of a beta-gamma C₁₀-C₄₄ alkyl olefin, gamma-delta C₁₀-C₄₄ alkyl olefin, delta-epsilon C₁₀-C₄₄ alkyl olefin, epsilon-zeta C₁₀-C₄₄ alkyl olefin, or zeta-eta C₁₀-C₄₄ alkyl olefin; and
ii. contacting the beta-gamma C₁₀-C₄₄ alkyl olefin, gamma-delta C₁₀-C₄₄ alkyl olefin, delta-epsilon C₁₀-C₄₄ alkyl olefin, epsilon-zeta C₁₀-C₄₄ alkyl olefin, or zeta-eta C₁₀-C₄₄ alkyl olefin product of step (i) with maleic anhydride under conditions suitable to provide a compound of the formula II 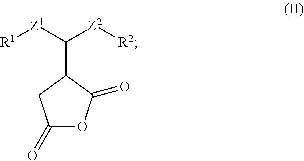
and
iii. contacting a compound of the formula II 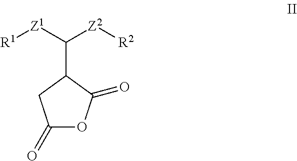
with an ammonia source under conditions sufficient to provide a compound of the formula I.

24. The process of any claim 19, wherein the catalyst used in step (i) is a solid acid catalyst, an aqueous acid catalyst, a supported metal catalyst, or an organometallic catalyst, or a combination of any one or more.

25. The process of claim 19, wherein the catalyst used in step (i) is sulfuric acid, p-toluenesulfonic acid, perfluorinated ion exchange resins (Nafion®), sulfonated poly(styrene-co-divinylbenzene) resins (PS-DVBs) including but not limited to Amberlyst 35, 70, XN1010, ZSM-35 and SAPO-11, tungstated zirconias, or acidic zeolites.

26. The process of claim 19, wherein the catalyst used in step (i) is Rhodium, Iridium, Nickel, Palladium, or Platinum.

27. The process of claim 19, wherein the catalyst used in step (i) is supported on carbon, aluminum oxide, titanium dioxide, silicon dioxide, zirconium dioxide, or magnesium oxide.

28. The process of claim 19, wherein step (i) is carried out at a temperature of from about 25° C. to about 250° C.

29. The process of claim 19, wherein the distribution of isomerized olefins in step (i) comprises one or more of a beta-gamma C₁₀-C₄₄ alkyl olefin, gamma-delta C₁₀-C₄₄ alkyl olefin, delta-epsilon C₁₀-C₄₄ alkyl olefin, epsilon-zeta C₁₀-C₄₄ alkyl olefin, or zeta-eta C₁₀-C₄₄ alkyl olefin.

30-61. (canceled)
